(19) United States
(12) Patent Application Publication
(10) Pub. No.: US 2026/0335176 A1
(43) Pub. Date: Oct. 29, 2026

(54) DEVICE FOR BEAUTY AND/OR WELLNESS TREATMENTS ON THE HUMAN BODY

(71) Applicant: B&M S.R.L., Milan (IT)

(72) Inventor: Sandro SANSONI, Ferrara (IT)

(73) Assignee: B&M S.R.L., Milan (IT)

(21) Appl. No.: 19/574,027

(22) Filed: Mar. 20, 2026

(30) Foreign Application Priority Data
Mar. 20, 2025 (IT) 102025000005760
Dec. 12, 2025 (IT) 102025000033052

(51) Int. Cl. A61H 15/02; A61H 15/00; A61N 5/06; A61N 5/067

(52) U.S. Cl. CPC A61H 15/02; A61H 15/0092; A61N 5/0616; A61N 5/067; A61H 2201/10; A61N 2005/0644; A61N 2005/0659

(57) ABSTRACT

An apparatus for beauty or wellness treatments includes a massaging device having a rotor adapted to be driven to rotate about a longitudinal axis and circumferentially provided with a plurality of massaging groups over a circular trajectory about the axis. Each massaging group includes a plurality of rotatable massaging elements arranged to roll on the patient's body, generating said pressure pulses on the patient's body. The apparatus includes a laser light emitter configured to irradiate: an area of the patient's body on which at least one of said massaging elements is rolling and/or on which at least one of said massaging elements is exerting a pressure pulse; and/or a first area of the patient's body adjacent to a second area of the patient's body on which at least one of said massaging elements is rolling and/or on which at least one of said massaging elements is exerting a pressure pulse.

The present claims priority of Italian patent applications No. IT102025000005760 and IT102025000033052, the contents of which are incorporates herein by reference.

### FIELD OF THE INVENTION

The present invention relates to an apparatus particularly suitable for performing beauty and/or wellness treatments for the purpose of toning or firming organic tissues such as, for example, skin, muscles, fat deposits and other soft tissues, as well as for toning muscles.

In particular, the apparatus according to the invention is intended for performing non-invasive beauty treatments, i.e. without the need for surgery.

### STATE OF THE ART

In the field of beauty treatments aimed at eliminating or reducing wrinkles and/or other skin imperfections, such as cellulite or other pathologies linked to defective blood circulation, as well as for toning the skin or muscles for wellness purposes, there are well-known devices (so-called “handpieces”) provided with devices with rotating balls, with a plurality of balls designed to interact with the skin with a vibrating action to produce a massaging effect on the skin itself, aimed in particular at reactivating the blood circulation and the consequent stimulation of the lymphatic system, improving cell oxygenation and helping to tone the muscles.

The general purpose of the present invention is to provide an apparatus for toning body tissues such as skin, muscles or fat masses, which is more effective than currently known apparatus.

### SUMMARY OF THE INVENTION

In view of this purpose, it is contemplated to provide, according to the invention, a beauty or wellness treatment apparatus according to claim 1.

The invention also relates to the use of such beauty and/or wellness treatment apparatus having the features of claim 19.

To clarify the explanation of the innovative principles of the present invention and the advantages thereof with respect to the prior art, a possible illustrative embodiment applying such principles will be described below, with the aid of the accompanying drawings.

### LIST OF FIGURES

In the drawings:

FIG. 1 shows an overall perspective view of an apparatus according to a first particular embodiment of the invention.

FIGS. 2, 3 and 4 show side elevation views of the apparatus in FIG. 1, taken from different angular positions around the longitudinal axis of the apparatus.

FIG. 5 shows a further perspective view of the apparatus in FIG. 1.

FIG. 6 shows an overall perspective view of an apparatus according to a second particular embodiment of the invention.

FIG. 7 shows a first side view of the apparatus in FIG. 6, according to the observation direction X;

FIG. 8 shows a second side view of the apparatus in FIG. 6, according to the observation direction Z;

FIG. 9 shows a partial sectional view, according to a sectional plane IX-IX parallel to the axes XZ, of the apparatus in FIG. 6.

### DETAILED DESCRIPTION

Unless otherwise specified, expressions such as “the quantity X has values comprised between A and B” are understood in this description to mean that the quantity X can take on not only intermediate values between A and B but also the value A and the value B.

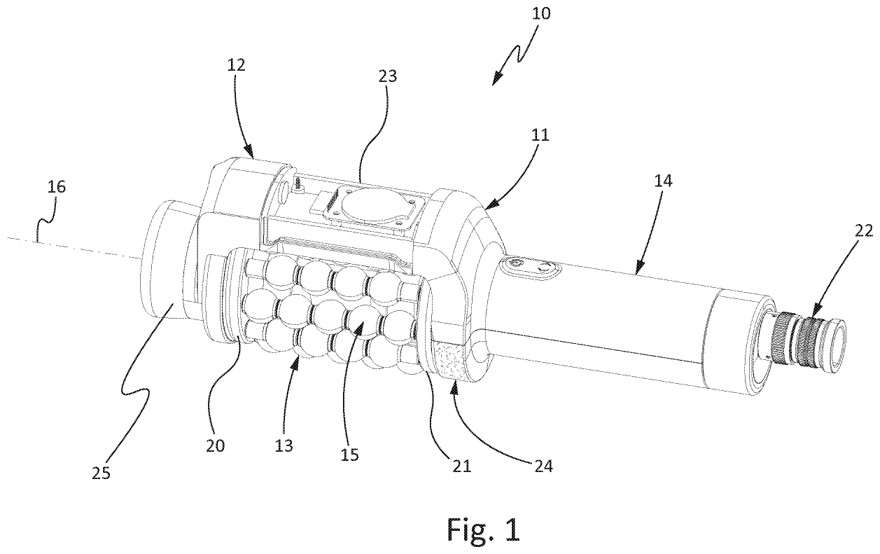

Referring to the figures, FIG. 1 shows in its entirety a beauty and/or wellness treatment apparatus 10, 10′ according to the invention.

The apparatus 10, 10′, in its most general lines, preferably comprises an outer shell 11 configured to form a front portion (or head portion) 12, provided with a massaging device 13 with rotating elements, and a rear portion (or first handgrip portion) 14, adapted to be gripped by the operator performing the treatment.

In this description, the apparatus 10, 10′ will also be referred to as a “handpiece”, a term by which these types of apparatuses are commonly known in this field of the art.

The massaging device 13 comprises a rotor 15 having an overall preferably cylindrical shape and driven in rotation about its longitudinal axis 16 by an electric motor, not visible in the present drawings, advantageously housed within the first handgrip portion 14.

The massaging device 13 is circumferentially provided with a plurality of massaging groups 17, each of which in turn comprises a plurality of massaging elements 18 preferably each aligned along its own axis of rotation 19 parallel to the axis of rotation 16 of the rotor 15.

The plurality of massaging groups 17 is preferably arranged around the axis of rotation 16 of the rotor 15 so as to substantially form a ring or bundle.

The massaging elements 18 of each massaging group 17 are advantageously in the form of balls and are rotatably mounted, e.g. idle, aligned on respective shafts (coinciding with said axes of rotation 19) fixed at their ends to a pair of support discs 20, 21 arranged at the two opposite ends of the cylindrical rotor 15.

At the rotating ball massaging device 13, the shell 11 has an opening in its circumferential wall, which extends angularly for an angular portion α of its circumferential development, so as to radially expose outwardly the massaging groups 17 with their respective massaging elements 18 and allow the latter to come into contact with the body of a subject to be treated.

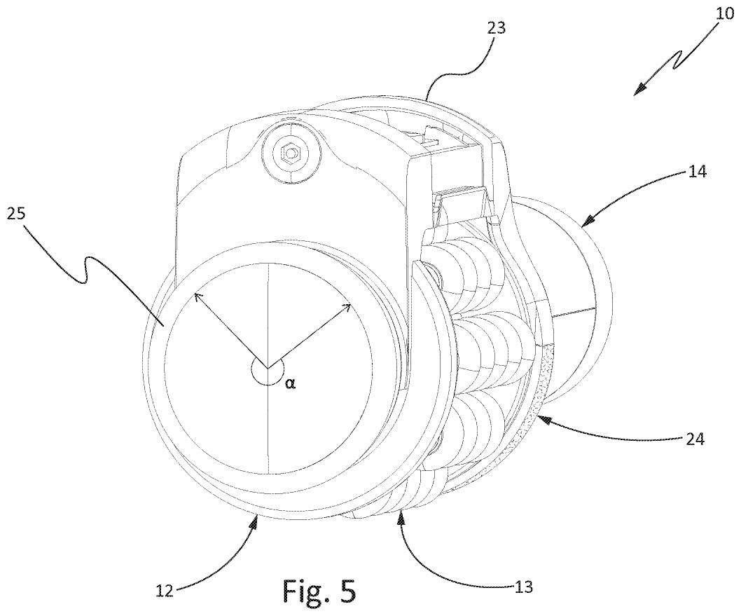

Advantageously, the sector or angular portion α (shown in FIG. 5) extends for at least 180°, more preferably for 240°-270°, for 200°-240° or for 205°-215° or 208°-212°.

The sector or angular portion & can advantageously extend for about 210°.

When the rotor 15 is driven to rotate by the motor about its axis 16, the support discs 20, 21, which are integral with the rotor, drive the massaging groups 17 to rotate along a circular trajectory of revolution about the axis 16 of the rotor.

When the handpiece is brought into contact with the patient's body, each massaging group 17 makes sequential contact with the surface of the body and, as the balls 18 are mounted idly on their shafts, the friction between the body and the balls causes the latter to rotate about their own axis 19.

The contact and, in particular, the sequential rolling of the balls of the various massaging groups 17 on the patient's body produces a sequence of pressure pulses on the body itself, generating a vibration that, as a person skilled in the art is well aware, is capable of having beneficial effects on the patient's skin and muscle tissues.

As for example in the embodiments described and illustrated herein, the rotor 15 may comprise ten massaging groups 17 evenly arranged around its circumference, with an alternating arrangement of groups of five balls and groups of four balls.

This alternating arrangement of an odd number and an even number of balls makes it possible to maximize the potential contact surface with the patient's body due to the fact that, as the balls of a massaging group with four balls are located in the space between two adjacent balls of the massaging groups with five balls, the density of outwardly exposed massaging balls can be increased.

The same result can naturally also be achieved by using massaging groups all with five balls, but alternately staggered axially, so that each ball of one massaging group lies in the space between two adjacent balls of the previous/next massaging group.

The apparatus 10, 10′ can be powered by batteries, or by mains power supply. In the latter case, as in the embodiment described and illustrated herein, a connector 22 will be provided (advantageously at the rear or first handgrip portion 14) for the power supply cables.

The apparatus 10, 10′ may also advantageously include an area 23 at the front or head portion 12, which is designed to accommodate controls, displays and other control devices, as is well known to a person skilled in the art.

In addition to the first handgrip 14, the apparatus 10 may possibly be provided with a second handgrip 25.

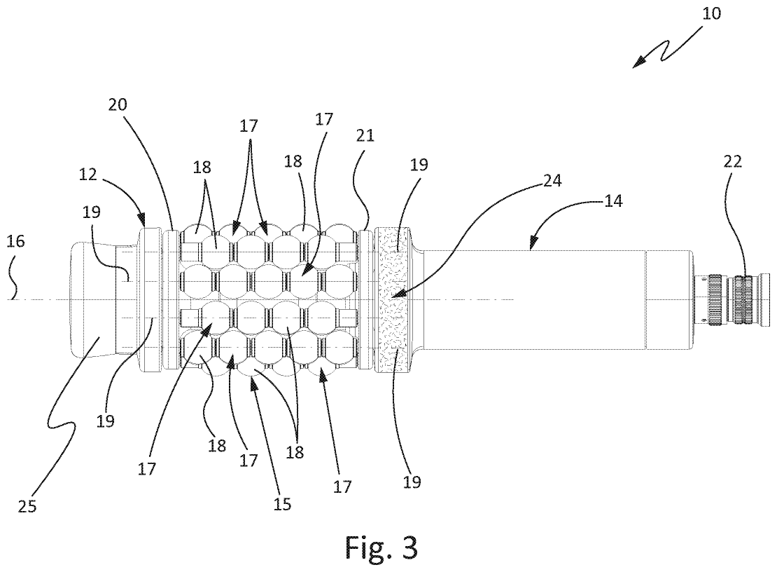

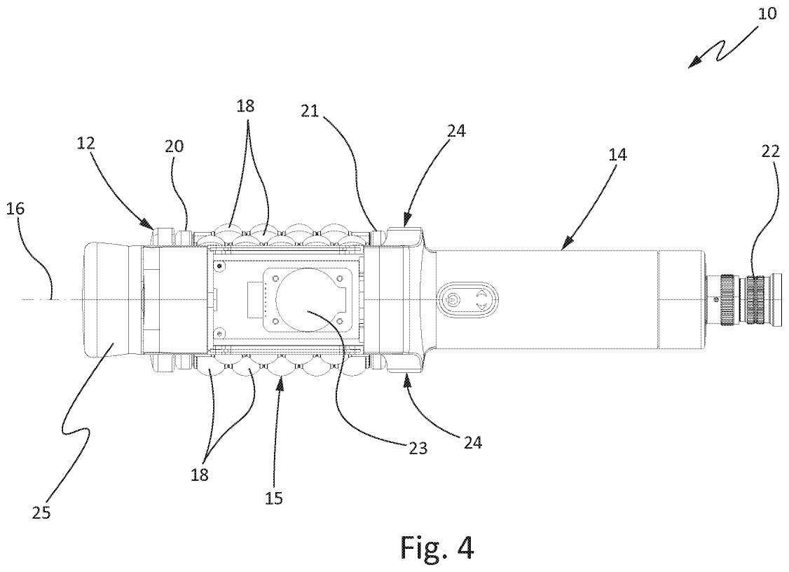

The second handgrip 25 is preferably placed on the side of the front or head portion 12, opposite the side to which the first handgrip portion 14 is attached (FIG. 1-4).

The first handgrip portion 14 and the second handgrip portion 25 are preferably and substantially coaxial to each other, and for example both coaxial to the axis of rotation 16 (FIG. 1-4) or to another longitudinal axis of the apparatus 10.

The second handgrip 25 preferably has an smaller axial extension than that of the first handgrip 14 (FIG. 1-4), so as to allow a user to hold the device 10 with both hands—that is, holding the handgrip 14 with one hand and the handgrip 25 with the other hand—and press the device 10 more forcefully against the body of the person to be treated, allowing the massaging balls of the device 13 to exert a more vigorous massage on the body of the person to be treated.

For this purpose, the second handgrip 25 may have an axial extension of, for example, one half, one third, one quarter, one fifth, one sixth or one eighth of that of the first handgrip 14.

The second handgrip 25 has an axial extension more preferably between 0.10-0.50 times, and even more preferably between 0.20-0.60 times, between one sixth and one quarter, between 0.20-0.25 times the axial extension of the first handgrip 14.

In order to be comfortably gripped although axially very short, the second handgrip 25 may have a greater mean or maximum diameter or mean or maximum width than the mean or maximum diameter or mean or maximum width of the first handgrip 14, wherein mean diameter or mean width means the arithmetic mean along the longitudinal axis of the handgrips 14, 25, of the diameters or other widths of the various cross sections of the handgrips 14, 25 respectively.

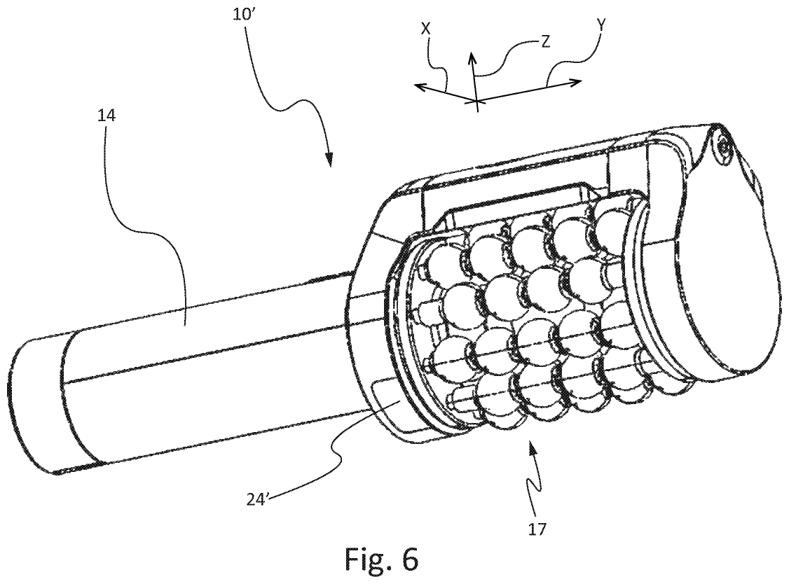

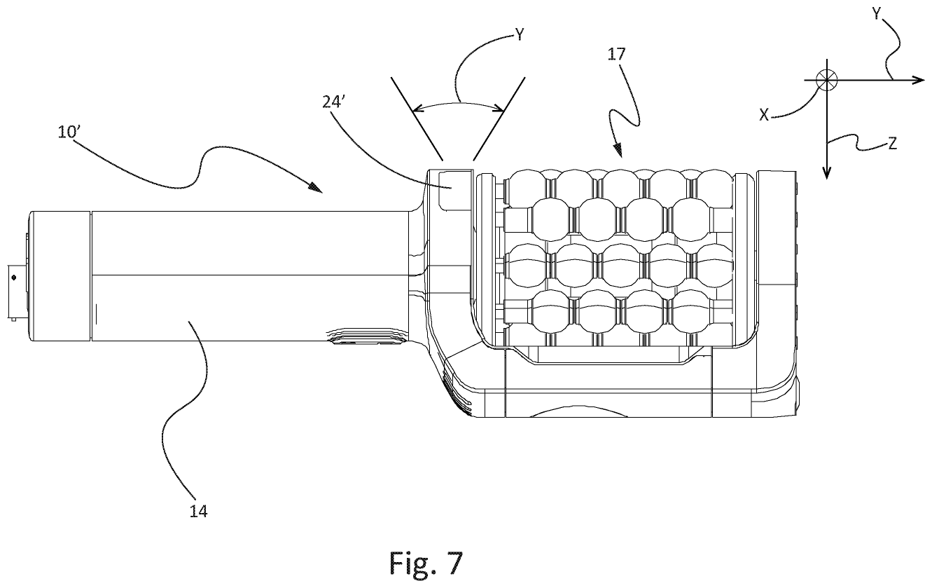

As for example in the particular embodiment of FIGS. 6, 7, the apparatus 10′ can also be devoid of the second handgrip 25 and be provided with, for example, only the first handgrip 14.

According to an innovative aspect peculiar to the present invention, the advantages of which will be more specifically set forth below, the apparatus 10, 10′ further comprises one or more light emitters 24, 24′, each of which is arranged to irradiate with light an area of the patient's body on which at least one of said massaging elements 18 of said massaging groups 17 is rolling and/or on which area at least one of said massaging elements 18 is exerting a pressure pulse, wherein said one or more emitters 24, 24′ may optionally be arranged to emit laser light.

Advantageously, the light emitter 24, 24′, which may optionally be a laser light emitter, is arranged at or near the massaging device 13, preferably, as in the embodiment described and illustrated herein, in the area between the massaging device 13 and the first handgrip portion 14.

Of course, if special construction and/or operating requirements should so require, the laser light emitter or non-laser light emitter could also be arranged at the opposite end of the massaging device 13, close to the front end of the apparatus 10, 10′.

Preferably, the light emitter 24, 24′ which may optionally be a laser light emitter, extends circumferentially along the side surface of the shell 11 for an angular portion equal to 180°, but could advantageously also extend less or more, for example for a length equal to the entire angular portion α of exposure of the massaging groups 17 and/or for an angular portion of amplitude for example comprised between 240°-270°, between 200°-240°, between 205°-215° or between 208°-212° or about equal to 210°.

Angular amplitude is preferably intended to mean the angular amplitude of an ideal dihedral whose edge coincides with the axis of rotation 16 of the rotor 15, or with the longitudinal axis of the first 14 or second handgrip 25 or another longitudinal axis of the apparatus 10.

The laser light emitter 24, 24′ is advantageously of the LLLT type, i.e. suitable for performing so-called “Low Level Laser Therapy”.

In particular, the laser light emitter 24, 24′ is arranged to emit laser light having one or more of the following characteristics:
wavelength greater than or equal to 600 nm, preferably comprised between 808 nm and 980 nm;
power such as to irradiate the skin of the subject to be treated, while in contact with at least one of said massaging elements 18, less than or equal to 40 mWatt/cm², preferable less than or equal to 20 mWatt/cm², to 10 mWatt/cm² or to 5.0 mWatt/cm².

The laser light emitter 24, 24′ can be arranged to emit laser light with power for example greater than or equal to 0.01 mwatt/cm², to 0.10 mWatt/cm², to 1.0 mWatt/cm² or to 2.0 mWatt/cm².

In another particular embodiment, one or more light emitters 24, 24′ are arranged to emit a non-laser light
substantially a red light in the visible band—having one or more of the following characteristics:
a wavelength comprised between 606-656 nm, or comprised between 616-646 nm, between 621-641 nm, between 623-639 nm, between 624-638 nm or between 626-636 nm;
power comprised between 0.10-2000 mw, or between 0.20-1000 mW, between 0.42-500 mw, between 1.0-210 mw, between 4.20-126 mW, between 21.0-84.0 mW, between 30.0-45.0 and e.g. approximately 40-42 mW.

Such non-laser light brings the benefits of, for example, chromotherapy or photobiostimulation, enhancing the toning effects of the rotatable massaging elements 18.

In yet another particular embodiment, the one or more light emitters 24, 24′ are arranged to emit non-laser infrared light having one or more of the following characteristics:
a wavelength comprised between 780-930 nm, or comprised between 810-900 nm, between 825-885 nm, between 830-880 nm or between 834-876 or between 840-870 nm;
power comprised between 3. 0 -3000 mW or between 10-1750 mw, between 35-1000 mW, between 170-700 mW, between 200-400 mw or approximately 350-370 mW.

This infrared light—or radiation—enhances the relaxing and invigorating effect of the rotatable massaging elements 18.

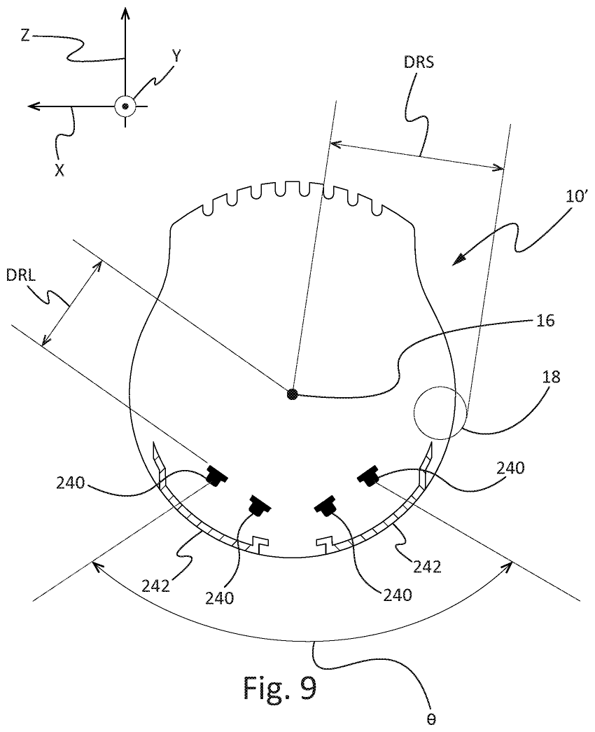

Each light emitter 24, 24′ which may optionally be a laser light emitter, may be arranged to emit a light beam—possibly of divergent laser light—in planes perpendicular to a longitudinal axis of the apparatus 10, for example perpendicular to the axis of rotation 16 or to the longitudinal axis of the first handgrip 14 and/or the second handgrip 25 with an angular aperture θ [theta], for example comprised between 1. 0°-270°, between 180°-270°, between 200°-240°, between 205°-215°, between 1.0°-10°, between 10-20°, between 20°-30°, between 8.00-40°, 8.0°-120°, between 10°-35°, between 40°-110°, between 70-78°, or for example about 210° (FIG. 9).

The angular aperture θ and the angle of divergence γ refer to the portion of the light beam in which the light intensity is greater than 50% of the peak intensity.

The wavelength and power of the laser or non-laser light will, of course, be selected according to the type and intensity of the treatment to be carried out, depending on the patient's pathology.

This type of laser has the ability to activate lipases, enzymes that can break down triglycerides into fatty acids and glycerol and thus promote the breakdown of excess fat.

In particular, it was found f that combining this type of laser or non-laser infrared or non-laser red with the vibration of the massaging device 13 significantly increased the effectiveness of the pulsing effect of the handpiece.

This is particularly useful in slimming treatments.

When using this apparatus to treat an area of a patient's body, the apparatus itself is repeatedly moved back and forth over the area to be treated, in contact with the patient's skin, thus combining the vibrating action of the ball massaging device 13 with simultaneous irradiation of the same area by the laser emitter.

The activation of lipases with their action against triglycerides enables the beneficial action already induced by the vibrating action of the massaging device on tissues affected by deficiencies in the blood circulation and lymphatic system to be further promoted and increased.

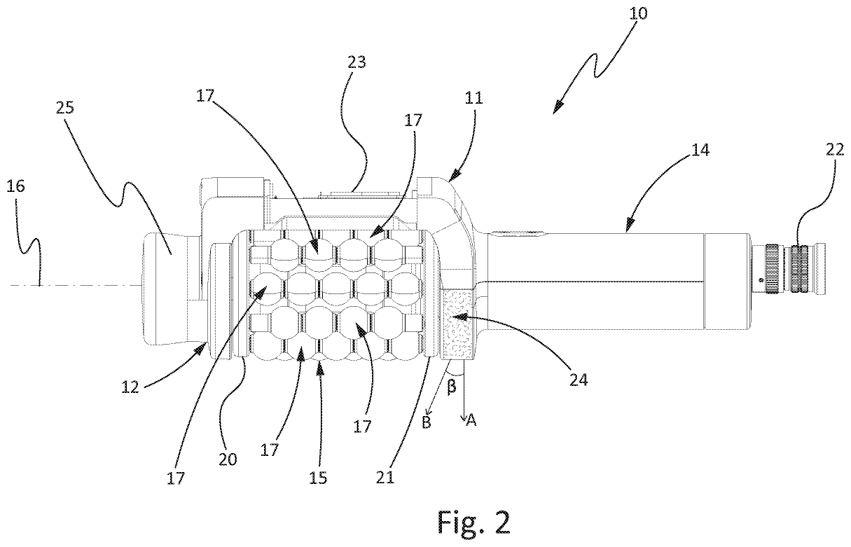

The emission of laser beams or non-laser light by the light emitter 24, 24′ can generally occur in a radial direction (i.e. perpendicular to the axis of rotation 16 of the rotor 15 of the massaging device 13), as indicated by arrow A in FIG. 2.

However, should it be desired to further perform the simultaneous action of the laser—and/or non-laser light—and the pressure pulses by the massaging elements 18, the emission of the laser or non-laser light beams could be enlarged with appropriate optical systems so as to direct it also towards the exposure area of the massaging groups 17, according to the arrow B shown in FIG. 2, thus with an emission direction forming an angle β [beta] preferably not zero with respect to the afore-mentioned radial direction A and for example comprised between 0.0°-40°, between 1.0°-20° or between 8.0°-35°.

The radial direction A is preferably perpendicular to the axis of rotation 16 and/or the longitudinal axis of the first handgrip 14.

Each emitter 24, 24′ can be arranged to emit a beam of laser or non-laser light with a divergence angle γ [gamma], in planes passing through the axis of rotation 16 and/or the longitudinal axis of the first handgrip 14, preferably comprised between 1.0°-50°, between 8.00-40° or between 10.0°-35° (FIG. 7).

Naturally, the foregoing description of certain embodiments that apply the innovative principles of the present invention is given as an example of such innovative principles and should therefore not be taken as limiting the scope of the invention claimed hereunder.

For example, the configuration of the rotor 15 with its massaging elements 18 may vary depending on the specific parts of the body to be treated for which the apparatus may be intended (e.g. face, legs, buttocks, etc.).

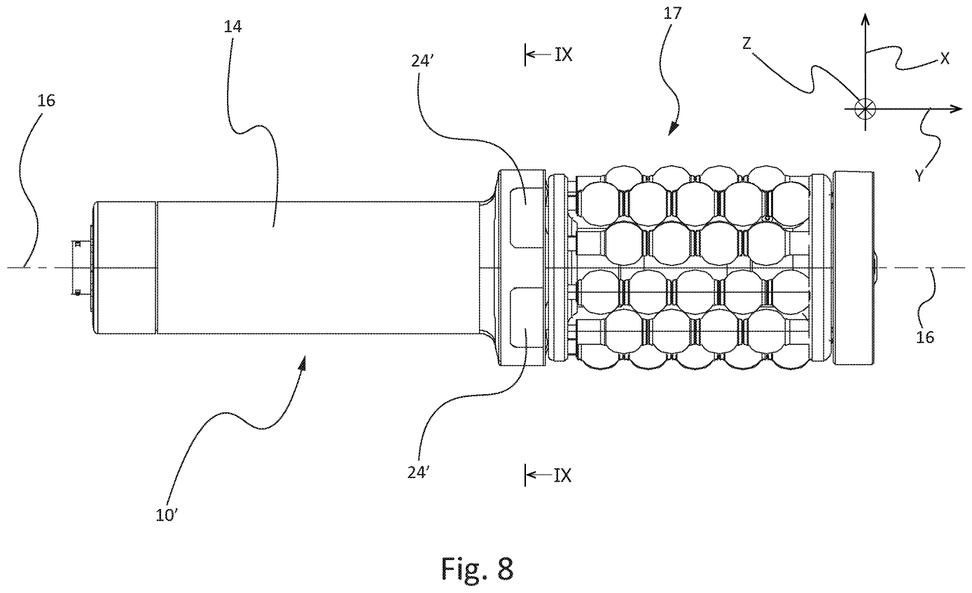

In a particular embodiment, an apparatus 10′ for beauty treatments can be equipped with two or more laser or non-laser light emitters 24′ (FIG. 6-9).

Each laser or non-laser light emitter 24, 24′ may comprise one or more LEDs (light-emitting diodes) 240, arranged for example in an arc or otherwise in a row or matrix, and/or a transparent protective shield 242 (FIG. 9).

Each LED 240 is arranged at a radial distance DRL from the axis of rotation 16 of the balls or other massaging elements 18, preferably comprised between 0.4-1.20 times the maximum radial distance DRS between the outer surface of one or more massaging elements 18 and the axis of rotation 16; the ratio DRL/DRS may also be comprised between, for example, 0.5-1.0 times, 0.6-0.9 times or 0.7-0.8 times.

In doing so, the LEDs 240 or other laser or non-laser light sources are very close to the skin of the subject to be treated, being approximately and generally during use about 10-30 millimetres from the patient's skin, increasing the energy of the laser or non-laser radiation hitting the patient's skin and the effectiveness of the treatment, as well as reducing the power consumption of the apparatus 10, 10′.

1) Beauty or wellness treatment apparatus, configured to be held by an operator, comprising:
a portion provided with a massaging device with rotating elements, which is at least partially exposed outwards and intended to produce pressure pulses on a patient's body, said massaging device in turn comprising a rotor adapted to be driven to rotate about a longitudinal axis of rotation thereof and circumferentially provided with a plurality of massaging groups intended to run, upon rotation of the rotor about its axis of rotation axis, over a circular trajectory about said axis of rotation of the rotor, each massaging group comprising, in turn, a plurality of rotatable massaging elements aligned along an axis of rotation thereof parallel to the axis of rotation of the rotor and arranged to rotate about their own axis of rotation rolling on the patient's body when contacting it, the massaging elements generating, upon contact with the patient's body, said pressure pulses on the patient's body,
wherein the beauty or wellness treatment apparatus further comprises, at or near the massaging device, a light emitter configured to irradiate with light:
A) an area of the patient's body on which at least one of said massaging elements is rolling and/or on which at least one of said massaging elements is exerting a pressure pulse; and/or
B) a first area of the patient's body adjacent to a second area of the patient's body on which at least one of said massaging elements is rolling and/or on which at least one of said massaging elements is exerting a pressure pulse.

2) The beauty or wellness treatment apparatus according to claim 1, wherein the laser light emitter is adapted to perform a ‘Low Level Laser Therapy’.

3) The beauty or wellness treatment apparatus according to claim 2, wherein the light emitter is configured to emit a laser light having one or more of the following features:
wavelength greater than or equal to 600 nm, preferably comprised between 808 nm and 980 nm;
power less than or equal to 40 m Watt/cm², preferably less than or equal to 10 mWatt/cm², during the irradiation of the area of the body to be treated while contacting at least one of said massaging elements.

4) The beauty or wellness treatment apparatus according to claim 1, wherein the light emitter is configured to emit a non-laser light having one or more of the following characteristics:
a wavelength comprised between 606-656 nm;
power comprised between 0.10-2000 mW.

5) The beauty or wellness treatment apparatus according to claim 1, wherein the light emitter is configured to emit a non-laser light having one or more of the following characteristics:
a wavelength comprised between 626-636 nm;
power comprised between 40-42 mW.

6) The beauty or wellness treatment apparatus according to claim 1, wherein the light emitter is configured to emit a non-laser light having one or more of the following characteristics:
a wavelength comprised between 780-930 nm;
power comprised between 3.0-3000 mW.

7) The beauty or wellness treatment apparatus according to claim 1, wherein the light emitter is configured to emit a non-laser light having one or more of the following characteristics:
a wavelength comprised between 840-870 nm;
power comprised between 350-370 mW.

8) The beauty or wellness treatment apparatus according to claim 1, wherein the rotor comprises ten massaging groups arranged along its circumference, for example evenly.

9) The beauty or wellness treatment apparatus according to claim 8, wherein the groups are present along the circumference of the rotor in an alternating arrangement of groups of five massaging elements and groups of four massaging elements.

10) The beauty or wellness treatment apparatus according to claim 1, wherein the massaging groups of the massaging device are exposed outwards of the apparatus, in order to contact the patient's body, for an angular portion around the axis of rotation of the rotor equal to at least 180°.

11) The beauty or wellness treatment apparatus according to claim 10, wherein the extension of said angular portion is within one of the following ranges:
240°-270°;
200°-240°;
205°-215°;
208°-212°.

12) The beauty or wellness treatment apparatus according to claim 1, wherein each light emitter is arranged to emit a beam of light divergent, in planes perpendicular to a longitudinal axis of the apparatus, with an angular aperture comprised between 1.0°-270°

13) The beauty or wellness treatment apparatus according to claim 1, wherein the light emitter extends circumferentially for an angular portion having one or more of the following amplitudes:
approximately equal to 180°;
comprised between 240°-270°;
comprised between 200°-240°;
comprised between 205°-215°;
comprised between 208°-212°.

14) The beauty or wellness treatment apparatus according to claim 10, wherein the light emitter extends circumferentially for an angular portion equal to the angular portion of exposure of the massaging groups.

15) The beauty or wellness treatment apparatus according to claim 1, further comprising a first handgrip and/or a second handgrip adapted to be held by an operator to use the beauty or wellness treatment apparatus itself.

16) The beauty or wellness treatment apparatus at least according to claim 15, further comprising only the first handgrip but not a second handgrip.

17) The beauty or wellness treatment apparatus according to claim 15, wherein the first handgrip has a substantially oblong overall shape.

18) The beauty or wellness treatment apparatus according to claim 17, wherein the second handgrip has an axial extension less than or equal to half the axial extension of the first handgrip.

19) A method of treating an area of a body of a patient, comprising:
providing the beauty or wellness treatment apparatus according to claim 1,
wherein said apparatus is moved repeatedly back and forth over an area of the body of the patient to be treated, in contact with the skin, combining the pressure pulses produced by the massaging device with a simultaneous irradiation of the same area of the body by the light emitter.

20) The beauty or wellness treatment apparatus according to claim 1, wherein the light emitter is configured to emit a laser light.
